(19) United States
(12) Patent Application Publication
(10) Pub. No.: US 2023/0277272 A1
(43) Pub. Date: Sep. 7, 2023

(54) In vivo large organ turnover and fixing device applied in minimally invasive surgery

(71) Applicant: Ning Fan, Qingdao (CN)

(72) Inventors: Ning Fan, Qingdao (CN); Chao Yang, Qingdao (CN); Bin Zhang, Qingdao (CN); Shuxian Wang, Qingdao (CN); Feng Wang, Qingdao (CN); Wei Rao, Qingdao (CN); Tianxiang Li, Qingdao (CN); Shan Liu, Qingdao (CN)

(21) Appl. No.: 17/809,550

(22) Filed: Jun. 28, 2022

(30) Foreign Application Priority Data
Mar. 2, 2022 (CN) 202210197533.8

(51) Int. Cl. A61B 90/17; A61B 17/02

(52) U.S. Cl. CPC A61B 90/17; A61B 17/0218; A61B 2017/0212

(57) ABSTRACT

The invention discloses an in vivo large organ turnover and fixing device applied in minimally invasive surgery, which comprises a device A and a device B which can be connected detachably, the device A comprises a connector I, a connecting rod I, a joint part and a control part I, the connector I is fixedly connected with the inner end of the connecting rod I, the control part I extends into the abdominal cavity through a minimally invasive hole on the abdominal cavity, the joint part is fixedly connected to the inner end of the control part I, and the movable end of the joint part is connected with the connecting rod I; the device B comprises a connector II, a connecting rod II and a control part II, the control part II extends into the abdominal cavity through a minimally invasive hole on the abdominal cavity, the connecting rod II is connected with the inner end of the control part II, and the connector II is fixedly connected with the connecting rod II. The invention relates to the technical field of surgical operating instruments, in particular to an in vivo large organ turnover and fixing device applied in minimally invasive surgery, which improves the turnover efficiency and precision of large organs under minimally invasive conditions and provides a guarantee for the minimally invasive surgery of liver transplant recipients.

### RELATED APPLICATIONS

This application claims priority to China Patent Application No. 202210197533.8, filed Mar. 2, 2022; the above-identified application is hereby incorporated by reference herein.

### TECHNICAL FIELD

The invention relates to the technical field of surgical operating instruments, in particular to an in vivo large organ turnover and fixing device applied in minimally invasive surgery.

### BACKGROUND ART

Under minimally invasive conditions (endoscopic and robotic surgery), operations are performed in the closed body space to turn over large organs such as liver, intestine cluster, and multi-organ collection of posterior peritonea (such as duodenum and pancreas) as a whole to expose the operation points fully. There are the following circumstances:
1) During an operation on livers, the right liver should be turned to the left to fully expose the right perihepatic ligaments so as to cut them;
2) To safely expose the tumor in the right posterior area of the liver to be resected under minimally invasive conditions, the liver needs to be fully turned over to the upper left of the liver;
3) In the minimally invasive liver transplant recipient surgery, the transplanted liver needs to be turned over to suture the blood oozing of each vascular anastomosis after the blood flow of the transplanted liver is restored.

For the above three circumstances, the current minimally invasive surgery only relies on the grasper to grip the liver or the gauze to remove the liver to constrainedly expose the operation point, which is often exposed poorly, resulting in subsidiary injury or poor hemostatic effect.

There are no minimally invasive devices that can hold large organs like the liver in place, so they can be turned over. The devices should have the following characteristics:
1) The devices are exquisite in design and occupy not much space to avoid blocking the view of minimally invasive surgery;
2) The devices have a large enough fixing range on the organ surface and enough adhesion to ensure the effective turnover of large organs in the body, and are not easy to slip;
3) The contact surface between the device and the large organ is soft, not easy to damage the organ, and maintains certain friction.

### SUMMARY OF THE INVENTION

Given the above problems, to overcome the defects of the prior art, the invention provides an in vivo large organ turnover and fixing device applied in minimally invasive surgery, which improves the turnover efficiency and precision of large organs under minimally invasive conditions and provides a guarantee for the minimally invasive surgery of liver transplant recipients.

The invention adopts the following technical proposal: an in vivo large organ turnover and fixing device applied in minimally invasive surgery in the proposal comprises a device A and a device B which can be connected detachably, the device A comprises a connector I, a connecting rod I, a joint part and a control part I, the connector I is fixedly connected with the inner end of the connecting rod I, the control part I extends into the abdominal cavity through a minimally invasive hole on the abdominal cavity, the joint part is fixedly connected to the inner end of the control part I, and the movable end of the joint part is connected with the connecting rod Ito realize the movable connection between the connecting rod I and the control part I through the joint part; the device B comprises a connector II, a connecting rod II and a control part II, the control part II extends into the abdominal cavity through the minimally invasive hole on the abdominal cavity, the connecting rod II is connected with the inner end of the control part II, and the connector II is fixedly connected with the connecting rod II and detachably hooked with the connector I.

As a further optimization of the scheme, the control part I comprises a control handle I and a connecting rod III, the connecting rod III extends to the abdominal cavity through a minimally invasive hole on the abdominal cavity and is fixedly connected with the joint part, and the control handle I is arranged at the outer end of the connecting rod III.

As the further preferred embodiment of the proposal, the control part II comprises a control handle II and a connecting rod IV, the connecting rod IV passes through the minimally invasive hole on the abdominal cavity and is clamped with the connecting rod II, and the control handle II is arranged on the outer end of the connecting rod IV.

Preferably, the connector I is provided with a one-way anti-off hook, the connector II is provided with a hook connecting ring, the one-way anti-off hook is unidirectionally open from the outside to the inside, the connector II is hooked with the hook connecting ring on the connector I in an anti-off way through a bayonet on the one-way anti-off hook, and the connector I and the connector II are not easily disconnected after being connected.

Preferably, the joint part is an electric joint structure, and the control handle I is provided with a joint rotation control button, which is electrically connected with the joint part to control the rotation angle of the connecting rod I relative to the connecting rod III, so that the connecting rod I is in contact with the surface of the large organ at different angles, thereby ensuring that the connecting rod I is tightly close to the organ when the organ is turned over, and preventing organ displacement or even slippage.

Preferably, a clamping slot is arranged at one end where the connecting rod II is away from the connector II, and an electric clamping head is fixedly arranged at the inner end of the connecting rod IV and is clamped with the clamping slot.

Preferably, a clamping control button is arranged on the control handle II and electrically connected with the electric clamping head to control the connection and disconnection between the electric clamping head and the clamping slot, so as to achieve the in vitro separation between the connecting rod II and the control part of the device B in vitro.

As the further preferred embodiment of the proposal, the connecting rod I and the connecting rod II are medical silicone rods with curvature on the surface, the surfaces of the connecting rod I and connecting rod II are the main surfaces in contact with the large organs, so the connecting rod I and connecting rod II are made of medical silicone materials with certain curvature, the surfaces are soft and has certain friction to achieve the function controlling the large organs such as a liver without damage to the organ surface, and the connecting rod I is 3-5 cm long.

As the further preferred embodiment of the proposal, the connecting head I and the connecting rod I are curved by 150-170 degrees.

As the further preferred embodiment of the proposal, the connecting head I, the connecting rod I, the joint part, the connecting head II, the connecting rod II, the connecting rod III, the connecting rod IV, the one-way anti-off hook, and the hook connecting ring are less than 5 mm in diameter to ensure that the parts extending into the abdominal cavity can pass through a 5 mm trocar hole.

The invention has the following beneficial effects by adopting the above structure: currently, there is no minimally invasive in vivo large organ turnover and fixing device. The proposal improves the turnover efficiency and precision of large organs under minimally invasive conditions, improves the operation efficiency of complex minimally invasive hepatobiliary surgeries, including minimally invasive surgery for right liver tumor and minimally invasive radical resection for hilar cholangiocarcinoma, makes for the free resection of the wide range of hepatic ligaments as well as the exposure and precise suture hemostasis of anastomotic stoma of the transplanted hepatic blood vessels, and provides a guarantee for the minimally invasive surgery of the liver transplantation recipient.

### DESCRIPTION OF THE DRAWINGS

The drawings are provided for further understanding of the invention. They form part of the specification and are used to explain the invention together with its embodiments and do not constitute a limitation on the invention. In the drawings:

FIG. 1 is the schematic diagram for the overall structure of an in vivo large organ turnover and fixing device applied in minimally invasive surgery in the proposal;

FIG. 2 is an enlarged drawing of part A of an in vivo large organ turnover and fixing device applied in minimally invasive surgery in the proposal.

Wherein, 1. Device A, 2. Device B, 3. Connector I, 4. Connecting rod I, 5. Joint part, 6. Control part I, 7. Connector II, 8. Connecting rod II, 9. Control part II, 10. Control handle I, 11. Connecting rod III, 12. Control handle II, 13. Connecting rod IV, 14. One-way anti-off hook, 15. Hooking connecting ring.

### EMBODIMENTS

The technical proposal in the embodiments of the invention is clearly and completely described below in combination with the drawings. Obviously, the described embodiments are only part of the embodiments of the invention, but not all embodiments. Based on the embodiments of the invention, all other embodiments obtained by ordinary technicians in the field without creative labor are within the scope of protection of the invention.

In the description of the invention, it should be understood that the orientation or position relations indicated by the terms such as “upper” and “lower,” “front” and “rear,” “left” and “right” “top” and “bottom” “inner” and “outer” are based on the orientation or position relations shown in the drawings for the convenience of describing the invention and simplifying the description. However, they do not indicate or imply that the device or component must have a particular orientation or be constructed and operated in a particular orientation, so they shall not be construed as a limitation on the invention.

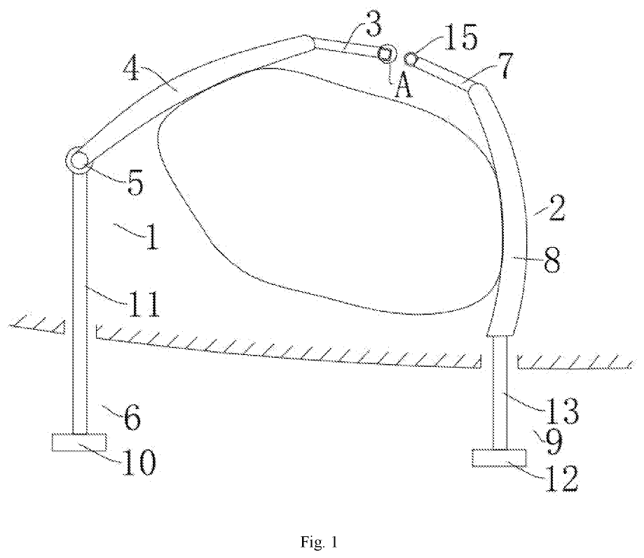

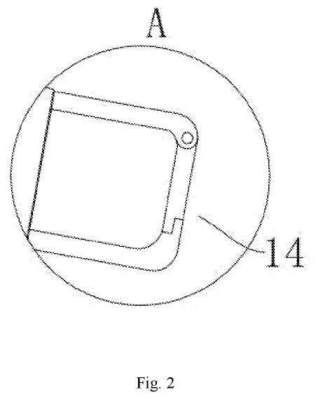

As shown in FIG. 1 and FIG. 2, an in vivo large organ turnover and fixing device applied in minimally invasive surgery in the proposal comprises a device A1 and a device B2 which can be connected detachably, the device A1 comprises a connector I 3, a connecting rod I 4, a joint part 5 and a control part I 6, the connector I 3 is fixedly connected with the inner end of the connecting rod I 4, the control part I 6 extends into the abdominal cavity through a minimally invasive hole on the abdominal cavity, the joint part 5 is fixedly connected to the inner end of the control part I 6, and the movable end of the joint part 5 is connected with the connecting rod I 4; the device B2 comprises a connector II 7, a connecting rod II 8 and a control part II 9, the control part II 9 extends into the abdominal cavity through the minimally invasive hole on the abdominal cavity, the connecting rod II 8 is connected with the inner end of the control part II 9, and the connector II 7 is fixedly connected with the connecting rod II 8 and detachably hooked with the connector I 3.

Wherein, the control part I 6 comprises a control handle I 10 and a connecting rod III 11, the connecting rod III 11 extends to the abdominal cavity through a minimally invasive hole on the abdominal cavity and is fixedly connected with the joint part 5, and the control handle I is arranged at the outer end of the connecting rod III 11. The control part II 9 comprises a control handle II 12 and a connecting rod IV 13, the connecting rod IV 13 passes through the minimally invasive hole on the abdominal cavity and is clamped with the connecting rod II 8, and the control handle II 12 is arranged on the outer end of the connecting rod IV 13.

The connector I 3 is provided with a one-way anti-off hook 14, the connector II 7 is provided with a hook connecting ring 15, and the one-way anti-off hook 14 is hooked with the hook connecting ring 15.

As shown in FIG. 1, the joint part 5 is an electric joint structure, and the control handle I 10 is provided with a joint rotation control button, which is electrically connected with the joint part 5.

Wherein a clamping slot is arranged at one end where the connecting rod II 8 is away from the connector II 7, an electric clamping head (not shown in the figure) is fixedly arranged at the inner end of the connecting rod IV 13, and is clamped with the clamping slot, a clamping control button is arranged on the control handle II 12 and electrically connected with the electric clamping head.

The connecting rod I 4 and the connecting rod II 8 are curved medical silicone rods, and the connecting rod I 4 is 3-5 cm long.

As shown in FIG. 1, the connecting head I 3 and the connecting rod I 4 are curved by 150-170 degrees.

Moreover, the connecting head I 3, the connecting rod I 4, the joint part 5, the connecting head II 7, the connecting rod II 8, the connecting rod III 11, the connecting rod IV 13, the one-way anti-off hook 14 and the hook connecting ring 15 are less than 5 mm in diameter.

The operation process of the device is as follows: The devices A1 and B2 are respectively assembled in vitro; after the devices A1 and B2 are put into the body, the joint part 5 is driven to rotate by adjusting the joint rotation control button on the control handle I 10 and drives the connecting rod I 4 to rotate to the right angle and ensure that the connecting rod I 4 is tightly close to the large organ, then the connector II 7 is clamped into the connector I 3 in on state, the connector I 3 is driven to be off through the hooking on-off control button on the control button I so that the devices A1 and B2 are connected in vivo, and the large organ is tightly close to the silicone surfaces of the connecting rod I 4 and the connecting rod II 8 to achieve the effective control of the large organ in vivo and guarantee that the large organ is precisely turned over to the operation location to precisely expose the operation point and complete the resection, suturing and hemostasis at the operation point;

Rapid removal of devices from the body at the end of surgery: As the connector I 3 is a one-way bayonet, the one-way anti-off hook 14 and the hook connecting ring 15 on the connector II 7 and the connector I 3 cannot be unlocked after interlocking, to complete precise turnover. After performing the surgical operation, place the large organ to the appropriate location, and start the quick removal operation, namely, the electric clamping head is driven to separate from the clamping slot through the clamping control button on the control handle II 12. The connecting rod IV 13 can be removed from the clamping slot in the connecting rod II 8 to the outside of the body, and the remaining in vivo part of the device B2 are removed from the other abdominal wall trocar hole along with the device A1.

It should be noted that the relational terms such as first and second in the text are only used to distinguish one entity or operation from another entity or operation and do not necessarily require or imply any such actual relationship or order between these entities or operations. Also, the terms “comprise” and “contain” or any other variants are intended to cover non-exclusive “contain,” so that the process, method, item, or device comprising a series of elements not only comprises those elements, but also other elements not listed clearly or inherent elements for the process, method or device.

The above shows and describes the basic principles and main characteristics of the invention and the advantages of the invention. For the technicians in the field, it is obvious that the invention is not limited to the details of the above demonstrative embodiments and can be realized in other concrete forms without deviation from the spirit or basic characteristics of the invention. Therefore, the embodiments should be considered exemplary and nonrestrictive for every point, and the scope of the invention is limited by the attached claims and not by the above description, so as to include all variations falling within the meaning and scope of the equivalent elements of the claims in the invention. No drawing reference signs in the claims shall be deemed to limit the claims involved.

In addition, it should be understood that the specification is described according to the embodiments, but not every embodiment only contains an independent technical proposal. The specification is described in such a narration way for clarity. The technicians in the field should take the specification as a whole. Technical proposals in the embodiments may be properly combined to form other embodiments that the technicians in the field can understand.

1. An in vivo large organ turnover and fixing device applied in minimally invasive surgery according to claim 1, which is characterized the in vivo large organ turnover and fixing device comprises a device A (1) and device B (2) which can be connected detachably, the device A (1) comprises a connector I (3), a connecting rod I (4), a joint part (5) and a control part I (6), the connector I (3) is fixedly connected with the inner end of the connecting rod I (4), the control part I (6) extends into the abdominal cavity through a minimally invasive hole on the abdominal cavity, the joint part (5) is fixedly connected to the inner end of the control part I (6), and the movable end of the joint part (5) is connected with the connecting rod I (4); the device B (2) comprises a connector II (7), a connecting rod II (8) and a control part II (9), the control part II (9) extends into the abdominal cavity through the minimally invasive hole on the abdominal cavity, the connecting rod II (8) is connected with the inner end of the control part II (9), and the connector II (7) is fixedly connected with the connecting rod II (8) and detachably hooked with the connector I (3).

2. An in vivo large organ turnover and fixing device applied in minimally invasive surgery according to claim 1, which is characterized in that the control part I (6) comprises a control handle I (10) and a connecting rod III (11), the connecting rod III (11) extends into the abdominal cavity through the minimally invasive hole on the abdominal cavity and is fixedly connected with the joint part (5), and the control handle I (10) is arranged on the outer end of the connecting rod III (11).

3. An in vivo large organ turnover and fixing device applied in minimally invasive surgery according to claim 2, which is characterized in that the control part II (9) comprises a control handle II (12), a connecting rod IV (13), the connecting rod IV (13) passes through the minimally invasive hole on the abdominal cavity and is clamped with the connecting rod II (8),and the control handle II (12) is arranged on the outer end of the connecting rod IV (13).

4. An in vivo large organ turnover and fixing device applied in minimally invasive surgery according to claim 3, which is characterized in that the connector I (3) is provided with a one-way anti-off hook (14), the connector II (7) is provided with a hook connecting ring (15), and the one-way anti-off hook (14) is unidirectionally open from the outside to the inside.

5. An in vivo large organ turnover and fixing device applied in minimally invasive surgery according to claim 2, which is characterized in that the joint part (5) is an electric joint structure, and the control handle I (10) is provided with a joint rotation control button, which is connected electrically with the joint part (5).

6. An in vivo large organ turnover and fixing device applied in minimally invasive surgery according to claim 3, which is characterized in that a clamping slot is arranged at one end where the connecting rod II (8) is away from the connector II (7), and an electric clamping head is fixedly arranged on the inner end of the connecting rod IV (13) and is clamped with the clamping slot.

7. An in vivo large organ turnover and fixing device applied in minimally invasive surgery according to claim 6, which is characterized in that a clamping control button is arranged on the control handle II (12) and is electrically connected with the electric clamping head.

8. An in vivo large organ turnover and fixing device applied in minimally invasive surgery according to claim 1, which is characterized in that the connecting rod I (4) and the connecting rod II (8) are medical silicone rods with curvature on the surface, and the connecting rod I (4) is 3-5 cm long.

9. An in vivo large organ turnover and fixing device applied in minimally invasive surgery according to claim 1, which is characterized in that the connecting head I (3) and the connecting rod I (4) are curved by 150-170 degrees.

10. An in vivo large organ turnover and fixing device applied in minimally invasive surgery according to claim 4, which is characterized in that the connecting head I (3), the connecting rod I (4), the joint part (5), the connecting head II (7), the connecting rod II (8), the connecting rod III (11), the connecting rod IV (13), the one-way anti-off hook (14) and the hook connecting ring (15) are less than 5 mm in diameter.
